(19) United States
(12) Patent Application Publication
(10) Pub. No.: US 2015/0366573 A1
(43) Pub. Date: Dec. 24, 2015

(54) INSTRUMENT, IN PARTICULAR MEDICAL-ENDOSCOPIC INSTRUMENT OR TECHNOSCOPE

(71) Applicant: RICHARD WOLF GMBH, Knittlingen (DE)

(72) Inventors: Friedrich HÄHNLE, Bretten (DE); Elmar TEICHTMANN, Bretten (DE); Frank WEHRHEIM, Bretten (DE); Matthias LAMBERTZ, Bretten (DE); Eberhard KÖRNER, Knittlingen (DE); Manfred BOEBEL, Neulingen-Bauschlott (DE)

(21) Appl. No.: 14/767,704

(22) PCT Filed: Feb. 4, 2014
(86) PCT No.: PCT/EP2014/052170

§ 371 Date: Aug. 13, 2015

(30) Foreign Application Priority Data
Feb. 15, 2013 (DE) 10 2013 202 503.3

(51) Int. Cl. A61B 17/29; A61B 19/00; A61B 17/00

(52) U.S. Cl. CPC A61B 17/29; A61B 17/00234; A61B 19/22; A61B 2017/00367; A61B 2019/2242; A61B 2017/2901; A61B 2017/2927; A61B 2017/2929; A61B 2017/2947

(57) ABSTRACT

An instrument, particularly a medical endoscopic instrument or technoscope, includes a shank (2) and an instrument head (6) which is arranged at a distal shank end. The instrument head (6) is bendable relative to the shank and includes a tool with two jaw parts (12, 14) which can be pivoted relative to one another. The instrument head (6), for controlling bending, is coupled in movement to at least one actuation rod (26). The actuation rod (26) is displaceably guided in the shank (2) in a shank longitudinal direction. For the control of the tool, the two jaw parts (12, 14) are coupled in movement to at least one pull cable which is led through the shank (2) to a proximal instrument end.

### CROSS REFERENCE TO RELATED APPLICATIONS

This application is a United States National Phase Application of International Application PCT/EP2014/052170 filed Feb. 4, 2014 and claims the benefit of priority under 35 U.S.C. §119 of German Patent Application 10 2013 202 503.3 filed Feb. 15, 2013 the entire contents of which are incorporated herein by reference.

### FIELD OF THE INVENTION

The invention relates to an instrument and in particular to a medical-endoscopic instrument or technoscope with a shank and with an instrument head which is arranged at the distal shank end, is bendable relative to the shank and comprises a tool with two jaw parts which can be pivoted relative to one another.

### BACKGROUND OF THE INVENTION

It is particularly in the field of medicine and there, in particular in the field of endoscopy, but also in other fields, for example for the application in cavities of technical objects, that shank instruments having an instrument head at the distal end of an elongate shank and with a tool arranged thereon, are applied. The control of these instruments, with which it can be the case e.g. of gripping instruments or cutting instruments, is usually effected proximally of the shank. For this, a suitably designed grip part is arranged there in the case of manually actuated instruments, and a control interface of a robotic system in the case of instruments which form part of a robotic system.

Such instruments, with which the instrument head can be bent relative to the shank and comprises a jaw tool, form the starting point of the invention. It is common to use pull cables for the control of the bending of the instrument head relative to the shank and for the control of the jaw parts of the jaw tool. These pull cables usually have the disadvantage that they are very prone to wear and must be led in the instruments in quite a complicated manner.

### SUMMARY OF THE INVENTION

Against this background, it is an object of the invention to provide an instrument, in particular a medical-endoscopic instrument or a technoscope, with an instrument head which can be bent (angled) relative to a shank and which is with a jaw tool arranged on it, said instrument having a more robust construction than the instruments of the type being discussed and which have been known until now, and being less complicated regarding manufacture.

With regard to the instrument according to the invention, it is particularly the case of a medical-endoscopic instrument or a technoscope. This instrument comprises a hollow, elongate shank which is preferably designed in a rigid manner, but as the case may be, can however also be flexible transverse to its longitudinal extension, at least in a part-region. An instrument head carrying a tool with two jaw parts which are pivotable relative to one another is arranged at the distal end of the shank. The instrument head can be bent relative to the shank.

The control of the bending of the instrument head and the control of the tool are effected from the proximal instrument end with the instrument according to the invention, wherein a control device for the control of the bending of the instrument head and of the tool which is arranged thereon is provided at the proximal side, which is to say proximally, of the shank. With regard to this control device, it can be the case of a handle for the manual control or of a control interface of a robotic system. The idea on which the invention is based lies in at least one pull cable which is coupled in movement to a jaw part and is led through the shank to the proximal instrument end being used in each case only for the force transmission from the control device onto the jaw parts and necessary for the control of the tool, whereas at least one actuation rod which is displaceably guided in the shank in the longitudinal direction of this and which is coupled in movement to the instrument head is used instead of at least one further pull cable, for the force transmission from the control device onto the instrument head and necessary for the control of the instrument head. The actuation rod which according to the invention is applied for the control of the bending of the instrument head and which is to be understood as any component which is suitable for transmitting pull and push forces and which can be led through the shank, has a significantly greater robustness and is accordingly less prone to wear, compared to the pull cables which have been used until now. The guidance of the actuation rod in the instrument is moreover significantly simpler than that of the pull cable.

A joint body, onto which a tool carrier distally connects and which engages into the distal shank end is advantageously formed on the instrument head of the instrument according to the invention, wherein the jaw parts of tool are pivotably articulated on the tool carrier about a pivot axis normal to the bending axis of the instrument head. The joint body is typically pivotable on the distal shank end about a joint axis or pivot which is formed there, i.e. pivotable transverse to the longitudinal axis of the shank. The outer contour of the joint body and the distal shank end are preferably designed in a manner such that the instrument head can be bent in an angular region of ±90° departing from a position, in which it is in the direct longitudinal extension of the shank. Preferably, one envisages the joint body having the shape of a ball, on which the spherical caps are cut away at sides which are directly away from one another. Usefully, the tool carrier connects to the joint body at a flat side of the ball body which is formed in this manner, whereas the actuation rod is articulated on the joint body at the other flat side which is designed in such a manner and which is arranged in the inside of the shank.

According to a further advantageous design of the instrument according to the invention, the jaw parts of the tool are articulated on two lateral sides of the tool carrier which are away from one another and are designed in a flattened manner. Accordingly, the tool carrier preferably comprises two flat sides which are directly away from one another, wherein in each case a jaw part is pivotable about a pivot axis aligned normally to the flat side, on each of these flat sides. The two flat sides formed on the tool carrier are usefully aligned normally to the bending plane of the instrument head, so that the tool or its jaw parts are pivotable in a plane which runs normally to the bending plane of the instrument head.

The instrument head further advantageously comprises a cavity which extends through this in the longitudinal direction and in which the pull cables are coupled in movement to the jaw parts. Hereby, the end of the joint body which engages into the shank is designed in an open manner, so that the pull cables led through the shank are led from there into the instrument head, where they are usefully coupled in movement to the two jaw parts, within the tool carrier. In this manner, the pull cables, departing from the proximal shank end, are arranged completely within the instrument according to the invention and are protected from external effects upon them, which in the most unfavourable case could lead to their damage and to a failure of the instrument.

Guide elements which lead the pull cables essentially through the pivot axis of the instrument head are advantageously arranged within the joint body of the instrument head, in order to prevent the bending of the instrument head from inadvertently affecting the alignment of the tool attached on the tool carrier or the alignment of its jaw parts. The effective length of the pull cables in the case of a bending of the instrument head is not influenced on account of this guidance of the pull cables through the pivot axis of the instrument head which is formed on the joint body, so that no pull forces which would otherwise lead to a pivoting of the jaw parts or of the tool, act upon the pull cables on account of the bending of the instrument head.

The pull cables according to a further advantageous development of the instrument according to the invention are fastened on wheels or wheel segments, which are arranged within the tool carrier and are coupled in movement to the jaw parts, for the control of the tool. The wheels or wheel segments are usefully each fastened in a rotationally fixed manner on a component which is rotatably mounted in the inside of the tool carrier and is coupled in movement to one of the jaw parts. The pull cables peripherally engage on the wheels or the wheel segments, i.e. in a manner radially distanced to the rotation axis of the component, on which the related wheel or wheel segment is fastened.

The two jaw parts of the tool can be coupled in movement to at least one pull cable led to the proximal shank end, in each case in a direct or indirect manner via suitable gear means. A cog gear can be advantageously provided for the movement coupling of the pull cables to the jaw parts. In this case, the pivot axes of the jaw parts are distanced to the rotation axes of the components, on which the pull cables are fastened preferably on wheels or wheel segments which are fastened thereto, wherein the wheels or wheel segments which are arranged within the tool carrier and on which the pull cables are fastened can advantageously be coupled in movement to cogs which mesh in each case with a toothing formed on a component connected in a rotationally fixed manner to a component forming the pivot axis of the jaw part.

The components themselves preferably form a part of the cog gear for the coupling of movement of the pull cables to the jaw parts. In this context, one preferably envisages the proximal end of the jaw parts in each case forming a cog segment which is meshed by a cog coupled in movement to the pull cable. Hereby, a toothing which is engaged with a cog arranged proximally of the jaw part and coupled in movement to at least one pull cable for the control of the jaw part is formed on a region which surrounds the pivot axis of the jaw part, is usefully designed in a peripherally circularly arcuate manner and is directed in the proximal direction, with each of the jaw parts. The cog segment of the two jaw parts can alternatively be also formed by a separate component which on the jaw part is inserted in a suitably designed recess. The cogs which are engaged with the toothing of the jaw parts, with the preferred arrangement of the jaw parts on lateral sides of the tool carrier which are designed in a flattened manner, are typically likewise arranged on these outer sides.

The instrument according to the invention has a particularly simple mechanical construction if the jaw parts are each directly coupled in movement to at least one pull cable, as is envisaged according to a further advantageous design of the instrument according to the invention. The movement coupling of the jaw parts in each case to at least one pull cable can be effected for example by way of a wheel or a wheel segment, on which the pull cable is fastened in a manner radially distanced to the pivot axis of the jaw part, being arranged preferably in the inside of the tool carrier, on a component forming the pivot axis of the jaw part.

However, a design with which two pins which are arranged parallel to the pivot axis of the jaw part, engage through an opening formed on the tool carrier and are connected to the pull cable within the tool carrier, are arranged on the sides of the jaw parts which face the tool carrier is preferred. Hereby, the opening formed on the tool carrier is usefully arranged in an annular manner around the pivot axis of the respective jaw part, wherein the two pins engaging into the opening are distanced to one another in the peripheral direction of the opening.

Particularly advantageously, the instrument head not only can be designed bendable relative to the shank, but additionally also be rotatable about its longitudinal axis relative to the shank. The range of application of the instrument according to the invention is increased and its operability is significantly simplified due to this measure.

The joint body of the instrument head is preferably designed in a spherical manner, in order to permit such a rotatability of the instrument head relative to the shank, wherein a groove which runs over the whole periphery of the joint body and into which two pins connected to the shank and forming the pivot axis of the instrument head is formed on the outer side of the joint body. The groove is formed on the joint body usefully where the joint body has its largest diameter. The groove, together with the pins which engage therein and which form the pivot axis of the joint body, forms a plain bearing whose sliding characteristics are improved by way of the groove and the end of the two pins which engage into the groove being designed in a suitably rounded manner.

The instrument head is advantageously connected to an actuation shaft which is rotatably mounted in the shank, in order to be able to rotate the instrument head relative to the shank, from the proximal side of the shank. This actuation shaft extends up to the proximal shank end, where, depending on the type of instrument, it is connected to actuation means of a handle which serve for rotating the instrument head or to a suitable control interface of a robotic system.

The actuation shaft is usefully not connected directly to the instrument head. Instead, the connection of the actuation shaft to the instrument head is preferably effected via the at least one actuation rod which is for bending the instrument head and is articulated on the joint body of the instrument head. Guides for the actuation rod and the pull cables for the control of the jaw parts, and which run through the actuation shaft in the longitudinal direction, are preferably formed on the actuation shaft. Accordingly, several guide channels which extend through the actuation shaft in the longitudinal direction and through which the actuation rod and the pull cables are led to the handle or control interface of a robotic system, which is located at the proximal shank end, are formed on the actuation shaft. The at least one guide channel for guiding the actuation rod has a cross section which is longitudinally widened in the direction of the bending plane of the instrument head, so that the actuation rod within the guide channel has the necessary space for its bending transverse to its longitudinal extension, since the bending of the instrument head also entails a certain bending of the actuation rod.

Preferably, with regard to the instrument according to the invention and for the control of the bending of the instrument head, two actuation rods are provided, and these are articulated on the proximal end of the instrument head at two sides of a pivot axis of the instrument head which are away from one another, in a manner distanced to this pivot axis. The bending of the instrument head is significantly simplified by way of the use of two actuation rods, as well as the rotatability of the instrument head relative to the shank in the case of an instrument head rotatable relative to the shank.

The invention is hereinafter explained in more detail by way of embodiment examples which are represented in the drawing. The drawings in a schematically simplified manner and in different scales The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective partly sectioned representation of an instrument;

FIG. 2 is a perspective partly sectioned representation of the instrument according to FIG. 1 with a bent or angled tool;

FIG. 3 is a perspective partly sectioned representation of the instrument according to FIG. 1 with a bent instrument head and bent tool;

FIG. 4 is a longitudinal sectioned representation of the instrument according to FIG. 1;

FIG. 5 is a perspective partly sectioned representation of an instrument according to a second embodiment; and

FIG. 6 is a longitudinal sectioned representation of the instrument according to FIG. 5.

### DESCRIPTION OF THE PREFERRED EMBODIMENTS

With regard to the instrument represented in the drawing in two embodiments, it is the case of a medical-endoscopic instrument in the form of a gripping forceps. This instrument comprises an elongate shank 2, wherein only the distal end of the shank 2 is represented in the drawing for reasons of an improved overview. The control device or the drives at the proximal end of the shank 2 are also not represented since these can be designed in a manner known per se.

The shank 2 is designed in a hollow-cylindrical manner, wherein projections 4 project in the axial direction of the shank 2, at the distal end of the shank at two diametrically opposite sides. The two projections 4 serve for the bendable articulation of an instrument head on the shank 2.

The instrument head 6 comprises a joint body 8, onto which a tool carrier 10 connects at the distal side, which is to say distally. The joint body 8 has the shape of a ball, on which the spherical caps have been omitted at two diametrically opposite sides. The tool carrier 10 which carries a tool with two jaw parts 12 and 14 which are pivotable relative to one another is arranged on one flat side which is formed on the joint body 8 in this manner.

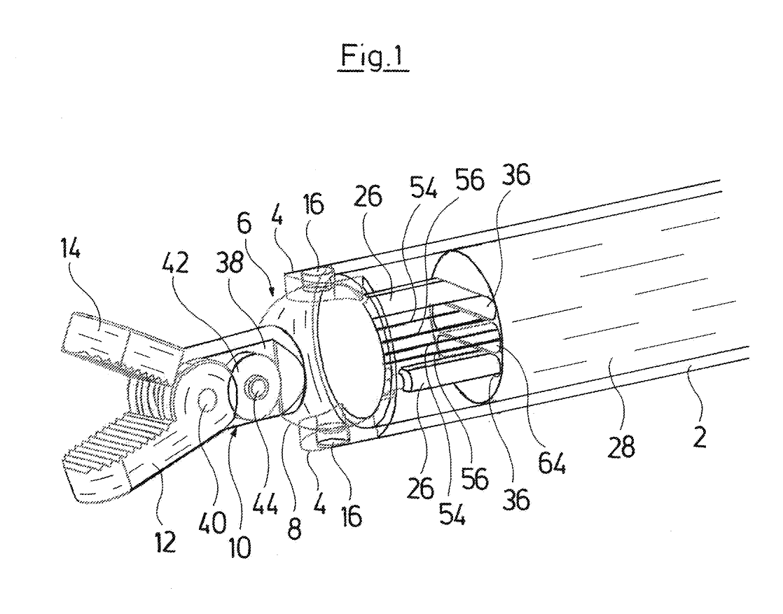

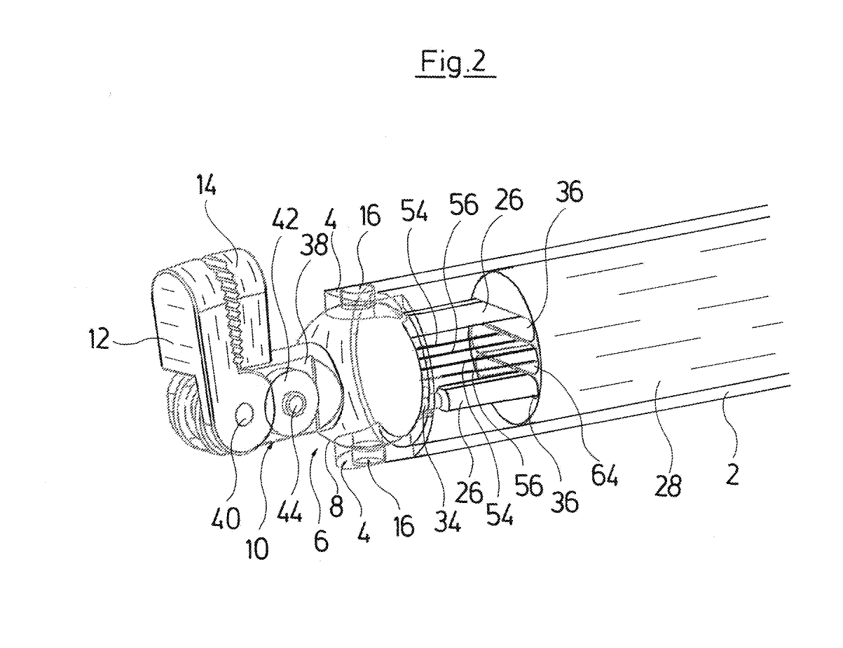

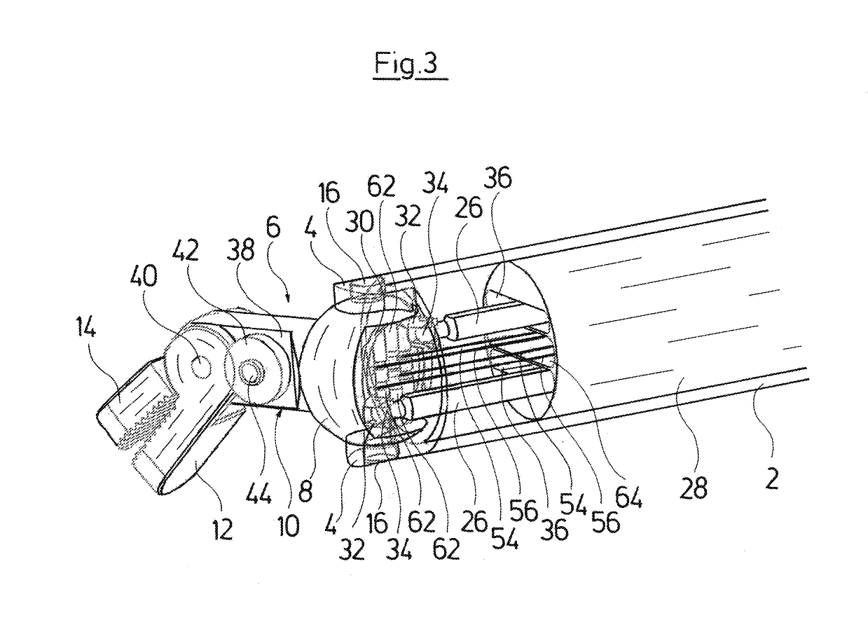

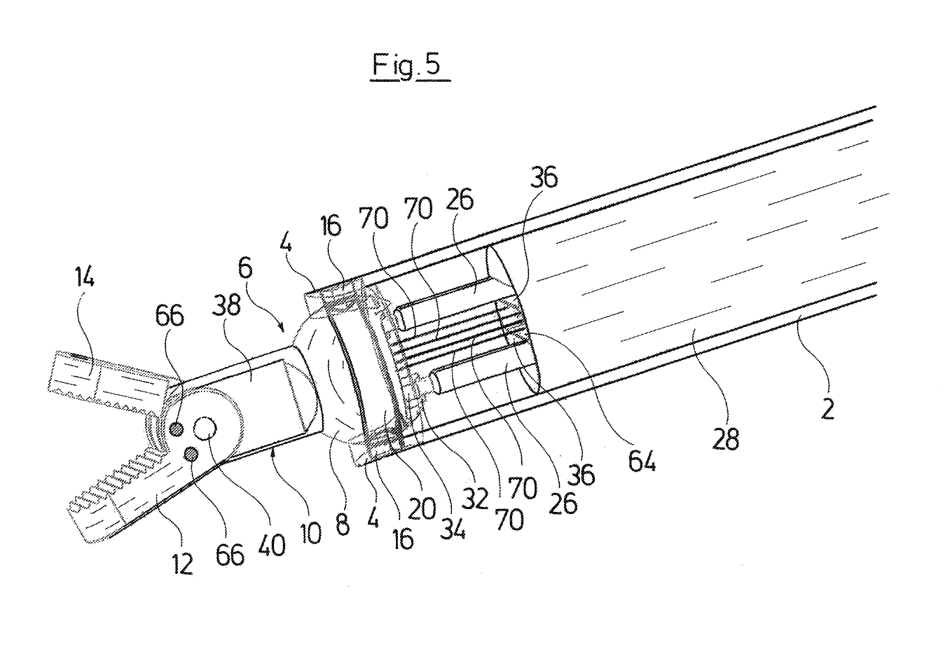

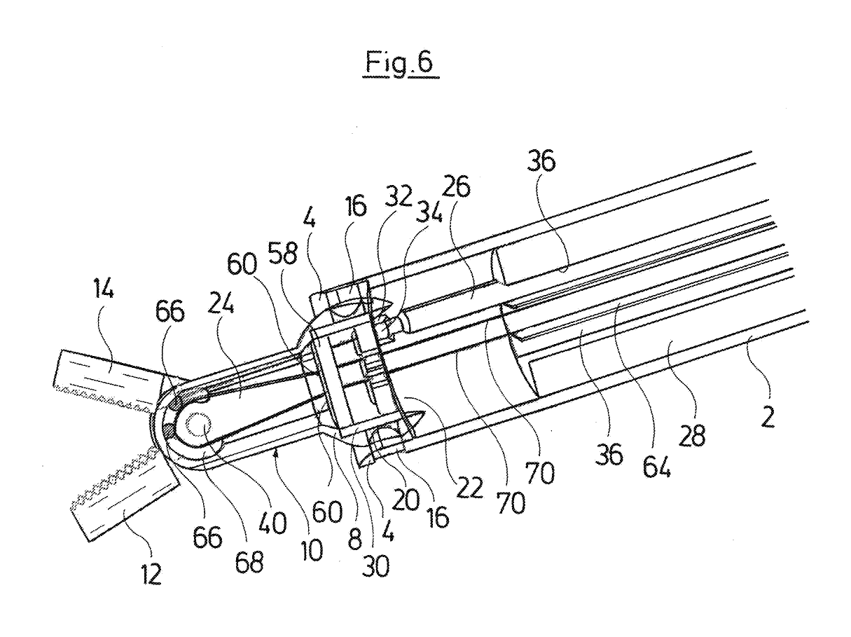

The instrument head 6 is articulated on the shaft 2 in a bendable manner via the joint body 8 of this instrument head. This articulation is effected via joint pins 16 which are led through openings formed on the projections 4 of the shank 2 and with the embodiment example represented in the FIGS. 1 to 4 engage in recesses 18 which are formed on the joint body 8 in the region of its largest diameter, at two locations on its outer side which are diametrically distanced to one another. With regard to the embodiment example represented in FIGS. 5 and 6, the joint pins 16 engage into a groove 20 which is formed on the joint body 8 and which on the joint body 8 in the region of its greatest diameter extends completely around the joint body 8. With the embodiment example represented in FIGS. 1 to 4 as well as with the embodiment example represented in FIGS. 5 and 6, the joint pins 16 form a pivot axis of the instrument head 6 which is aligned normally to the longitudinal extension of the shank 2.

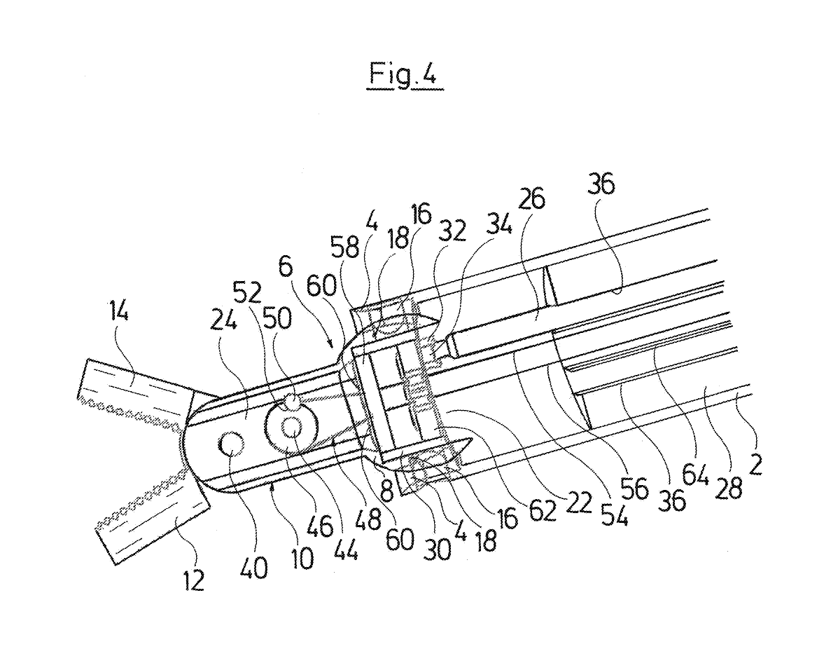

The instrument head 6 is designed in a hollow manner, as is particularly evident in particular from FIGS. 4 and 6. Thus a cylindrical cavity 22, onto which a cavity 24 formed in the tool carrier 10 connects distally, is formed on the joint body 8 in a manner departing from the proximal end of this joint body.

Two actuation rods 26 which are led through the shank 2 and there, through an actuation shaft 28 arranged in the shank 2, to the proximal shank end where they are coupled in movement to a control device of a handle to be operated manually or of a robotic system, serve for the control of the bending of the instrument head 6 relative to the shank 2.

A hollow-cylindrical sleeve 30 whose outer dimensions correspond to the dimensions of the cavity 22 is inserted into the cavity 22 of the joint body 22. Two mounting elements 32 are arranged on two opposite sides of the pivot axis of the instrument head 6 which is formed by the joint pins 16, on the outer edge of the sleeve 30, at the proximal end of the sleeve 30. In each case, one of the actuation rods 26 is articulated on these mounting elements 32 via joint heads 34 formed on the distal end of the actuation rods 26. For bending the instrument head 6, one of the two actuation rods 26 is displaced in the distal direction, whereas the other actuation rod 26 is displaced in the proximal direction in a corresponding manner. The instrument head can be bent in an angular range of ±90°, departing from an alignment represented in the FIGS. 1, 4, 5 and 6, in which it is aligned in the direct longitudinal extension of the shank 2. Hereby, the actuation rods 26 also undergo a slight bending transverse to their longitudinal axis. The actuation rods 26 are led in the actuation shaft in guides 36, whose cross section is longitudinally widened in the direction of the bending plane of the instrument head 6, in order to permit this bending of the actuation rods 26.

Two lateral sides 38 which are directly away, which is to say distant to one another, are designed in a flattened manner on the tool carrier 10. One of the jaw parts 12 and 14 is articulated on each of these lateral sides 38. The articulation of the jaw parts 12 and 14 on the tool carrier 10 is effected via joint pins 40. One joint pin 40 is connected in a rotationally fixed manner to one of the jaw parts 12 and 14 in each case. The joint pins 40 with their end which is away from the jaw part 12 and 14 engages into the cavity 24 of the tool carrier 10 via holes which are formed on the two lateral sides 38. The joint pins 40 are rotatably mounted in the holes which are formed on the two lateral sides 38 and which have a common middle axis, so that the two jaw parts 12 and 14 are pivotable about a pivot axis which is formed by the joint pins 40.

With regard to the instrument which is represented in FIGS. 1 to 4, a proximal end region of the jaw parts 12 and 14 which surrounds the joint pin 40 is designed in a circularly rounded manner. The jaw parts 12 and 14 in this proximal end region comprise a toothing which is not represented in the drawing for reasons of a better overview. This toothing of the jaw parts 12 and 14 in each case meshes with a cog 42 which is arranged on the lateral side 38 proximally of the jaw part 12 or 14. The cog 42 is connected in a rotationally fixed manner to a joint pin 44 which via a further hole formed on the lateral side 38 engages into the cavity 24 of the tool carrier 10. The rotatably mounted joint pin 44 forms the rotation axis of the cog 42.

A wheel 46 is connected to the joint pin 44 (FIG. 4) in a rotationally fixed manner, on the section of the joint pins 44 which engages into the cavity 24 of the tool carrier 10. The wheels 46 which are connected to the joint pins 44 are each distally wrapped by a pull cable 48. Hereby, the pull cables 48 are fixedly connected to the wheels 46 via fastening elements 50 which are fixed in recesses 52 formed on the outer periphery of the wheels 46. The ends 54 and 56 of the pull cables 48 are led through the instrument head 6 and the shank 2 to the proximal shank end, where they are coupled in movement to a control device. If one of the two ends 54 and 56 of the pull cables 48 is loaded in tension by way of the control device, then the wheel 46 which is connected to the respective pull cable 48 is rotated by way of this, and the jaw part 12 or 14 which is connected thereto is pivoted.

The sleeve 30 which is arranged in the cavity 22 of the joint body 8 is closed by a plate 58 at its distal end. Four openings 60, through which the ends 54 and 56 of the two pull cables 48 are led, are formed in a paired manner next to one another on this plate 58. Three, selectively also four essentially cylindrical guide elements 62 are arranged in the sleeve 30, proximally of the plate 58. The ends 54 and 56 of a pull cable 48 are led in each case between two guide elements 62 arranged directly next to one another. Two peripheral guide grooves are formed on the guide elements 62 next to one another in its longitudinal direction for this. The arrangement of the guide elements 62 in the sleeve is of such a nature that the ends 54 and 56 of the two pull cables 48 are led through the pivot axis of the instrument head 6 which is formed by the joint pins 16, given an instrument head 6 bent away relative to the shank 2. The effect of this is that the effective length of the pull cables 48 is not influenced in the case of a bending of the instrument head 6, so that no tensile forces arise on the pull cables 48 due to the bending of the instrument head 6, wherein these tensile forces would otherwise lead to a pivoting of the jaw parts 12 and 14. The ends 54 and 56 of the two pull cables 48 are led together in a guide channel 64 which is arranged between the two guides 36 for the actuation rods 26, in the actuation shaft 28 arranged in the shank 2.

With regard to the instrument represented in FIGS. 5 and 6, the jaw parts 12 and 14 are coupled in movement directly to the pull cables for their control. For this, two pins 66 which project on that side of the jaw parts 12 and 14 which faces the lateral side 38 of the tool carrier 10 are arranged on the jaw parts 12 and 14 radially on the outer side of the joint pins 40 connected to the jaw parts 12 and 14. The two pins 66 of each jaw part 12 or 14 engage through an opening 68 which is formed on the two lateral sides 38 of the tool carrier 10 and which extends annularly around the joint pin 40. A pull cable 70 for controlling the jaw part 12 and 14 is fastened at the two ends of the pins 66 which engage through the openings 68 into the cavity 24 of the tool carrier 10, and this pull cable is led to the proximal end of the shank 2 as with the instrument represented in the FIGS. 1 to 4.

In contrast to the instrument represented in FIGS. 1 to 4, the instrument head 6 is rotatable relative to the shank 2 with the instrument represented in FIGS. 5 and 6. This permits the groove 20 which is formed on the joint body 8 of the instrument head 6 and into which the joint pins 16 forming the pivot axis of the instrument head 6 engage. The actuation shaft 28 serves for the control of the rotation movement of the instrument head 6 and for this purpose is rotatably mounted in the shank 2.

While specific embodiments of the invention have been shown and described in detail to illustrate the application of the principles of the invention, it will be understood that the invention may be embodied otherwise without departing from such principles.

1. An instrument comprising:
a shank
an instrument head arranged at a distal shank end that is bendable relative to the shank and comprises a tool with two jaw parts which can be pivoted relative to one another;
at least one actuation rod, wherein the instrument head for the control of bending is coupled in movement to the at least one actuation rod which is displaceably guided in the shank in a longitudinal direction of the shank; and
pull cables, wherein each of the jaw parts is coupled in movement to at least one of the pull cables which is led through the shank to the proximal instrument end, for the control of the tool.

2. An instrument according to claim 1, wherein the instrument head further comprises:
a joint body which engages into the distal shank end and
a tool carrier on which the jaw parts of the tool are pivotably articulated about a pivot axis normal to a pivot axis of the instrument head, the tool carrier being connected to a distal portion of the joint body.

3. An instrument according to claim 2, wherein the jaw parts of the tool are articulated on two lateral sides of the tool carrier which lateral sides are away from one another and are designed in a flattened manner.

4. An instrument according to claim 1, wherein the instrument head comprises a cavity which extends in the longitudinal direction through this instrument head and in which the pull cables are coupled in movement to the jaw parts.

5. An instrument according to claim 4, wherein: the instrument head further comprises:
a joint body;
guide elements arranged within the joint body of the instrument head, said guide elements leading the pull cables essentially through a pivot of the instrument head.

6. An instrument according to claim 2, wherein the pull cables are fastened on wheels or wheel segments, which are arranged within the tool carrier and which are coupled in movement to the jaw parts.

7. An instrument according to claim 1, further comprising a cog gear, wherein the jaw parts are coupled in movement to a respective one of the pull cables via the cog gear.

8. An instrument according to claim 7, wherein the proximal end of the jaw parts in each case forms a cog segment which is meshed by a cog coupled in movement to a respective one of the pull cables.

9. An instrument according to claim 8, wherein the cogs are each coupled in movement to one of the wheels arranged within the tool carrier.

10. An instrument according to claim 1, wherein the jaw parts are each directly coupled in movement to at least one of the pull cables.

11. An instrument according to claim 10, wherein the instrument head further comprises a tool carrier on which the jaw parts of the tool are pivotably articulated about a pivot axis normal to the pivot axis of the instrument and two pins which are aligned parallel to the pivot axis of the jaw part, and engage through respective openings formed on the tool carrier and are connected within the tool carrier to a respective one of the pull cables, the pins being arranged in each case on the sides of the jaw parts which face the tool carrier.

12. An instrument according to claim 1, wherein the instrument head is rotatable about an instrument head longitudinal axis relative to the shank.

13. An instrument according to claim 2, wherein the joint body of the instrument head is designed in a spherical manner, wherein a groove which runs around the complete periphery of the joint body and into which two pins connected to the shank and forming the pivot of the instrument head engage, is formed on the outer side of the joint body.

14. An instrument according to claim 13, further comprising an actuation shaft wherein the instrument head is connected to the actuation shaft, which is rotatably mounted in the shank.

15. An instrument according to claim 14, wherein guides for the at least one actuation rod for bending the instrument head and for the pull cables for the control of the jaw parts are formed on the actuation shaft in a manner running through this in the longitudinal direction.

16. An instrument according to claim 1, further comprising two actuation rods which are articulated on the proximal end of the instrument head at two sides of a pivot axis of the instrument head which are away from one another are provided for the control of the bending of the instrument head.
